(19) United States
(12) Patent Application Publication
(10) Pub. No.: US 2010/0310615 A1
(43) Pub. Date: Dec. 9, 2010

(54) COMPOSITIONS COMPRISING STILBENE POLYPHENOL DERIVATIVES AND USE THEREOF FOR COMBATING THE AGEING OF LIVING ORGANISMS AND DISEASES AFFECTING SAME

(76) Inventor: Joseph Vercauteren, Castelnau-le-lez (FR)

Correspondence Address: NIXON & VANDERHYE, PC, 901 NORTH GLEBE ROAD, 11TH FLOOR, ARLINGTON, VA, 22203, US

(21) Appl. No.: 12/734,677

(22) PCT Filed: Nov. 17, 2008
(86) PCT No.: PCT/IB2008/054818

§ 371 Date: May 14, 2010

(30) Foreign Application Priority Data
Nov. 15, 2007 (FR) 07/08020

(51) Int. Cl. A61K 8/37; C07C 69/527; C07C 69/30; C07C 69/533; C07C 43/205; C07C 43/215; C07D 407/10; C07D 307/80; A61K 8/72; A61K 8/14; A61Q 19/08; C08G 65/38; C08F 12/24

(52) U.S. Cl. 424/401; 560/138; 560/144; 568/644; 568/646; 549/469; 560/141; 514/548; 514/549; 514/552; 424/78.31; 424/78.37; 424/78.03; 528/219; 526/313

(57) ABSTRACT

The invention relates to compositions comprising pholyphenol derivatives, characterised in that said polyphenols contain monomers, oligomers or polymers with units having formula (I), said units being characterised by the simultaneous presence of a resorcinol nucleus (nucleus A) and a para-phenol nucleus (nucleus B) which are interconnected by a carbon bond C, said derivatives being over-activated, in respect of the nucleophilic power thereof, by alkylation of at least one phenol function of each constituent monomer unit and stabilised by sterification by mixtures of fatty acids in proportions reflecting those of vegetable oils formed mainly by unsaturated fatty acids of all of the other phenol functions. The invention is suitable for use in cosmetics, dietetics and therapeutics.

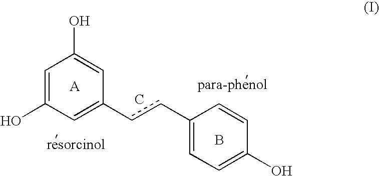

The invention relates to compositions of stilbene polyphenol derivatives for preventing and controlling the majority of pathologies and the aging of living organisms and tissues. It also relates to a process for preparing these compositions, and to their applications, especially in the fields of cosmetology, dietetics, and therapeutics.

More than fifty years ago, a theory developed whereby the aging of the human body is a result of the accumulation of multiple damage caused to the tissues by free-radical species or oxidizing chemical reactivities.

In the middle of the 1950s, after numerous studies on rubber, the chemist Harman observed that preventing the formation of free radicals was the most certain way to prevent its degradation and cracking. By analogy, he then suggested that the aging of human tissues (appearance of wrinkles in the skin, for example) might be caused by the “abnormal” formation, within cells, of highly reactive chemical species, and especially free radicals, and by the reaction sequences that they triggered.

Reactive oxygen species (ROS) are formed at the mitochondrial level by uncontrolled “transfer” of one or more electrons to oxygen (ROS: superoxide anion, peroxides, peroxynitrites, free radicals, etc.).

These ROS subsequently propagate to the other cellular compartments or to the cytoplasm, depending on their water/fat solubility, where they produce considerable damage.

In this kind of context, the search for active substances for controlling aging has been conducted, over the recent decades, on the basis of their capacity to break the chain oxidation reactions, in other words to prevent the oxidative stress. In effect, any substance capable of interacting with the ROS will lessen the deleterious effects and, over the longer term, will have a positive impact on health, and, for the same reasons, will slow down aging and the development of the main pathologies. Such substances are free radical scavengers (capable of delivering a single electron at a time) and/or antioxidants (transfer of two electrons at the same time) such as vitamins (E and C) and polyphenols.

However, the damage caused by the aging of the body or accompanying the major pathologies is unlikely to be solely the consequence of poor control of the flow of electrons owing to “leaks” of the mitochondrial metabolism and of intracellular ROS, but is also likely to involve other sources of potential deleterious effects, involving the Maillard reaction and carbonyl stress.

In carbonyl stress, the carbonyl (aldehyde) function of glucose exerts its electrophilic properties with regard to the nucleophilic residues of proteins (amines, thiols, etc.): this is the starting point for carbonyl stress, which is amplified by formation of propagators.

The chemical species produced, or glycation products, are considered to be end products: these are AGES, for Advanced Glycated End-Products, in which glucose or its fragments are joined irreversibly to the amino acid residues.

The Maillard reactions which take place increase, at the same time, the reducing capacity of the sugars and of their derivatives. The dicarbonyl compounds which form acquire an oxidizability which is much greater even then their precursors, and readily transfer their electrons to oxygen, for example. Starting from the superoxide anion formed initially, the same sequence of ROS as in the case of intracellular stress is produced. Accordingly, the carbonyl stress is coupled with a second type of an oxidative stress.

In contradistinction to the mechanisms set out above for the ROS of mitochondrial origin, this new oxidative stress occurs outside the cells, within the extracellular matrix. It therefore affects the amino acids or protein residues of this matrix, and especially the fibers of collagen and of elastin. This oxidative stress, which is particularly significant in view of the fact that the enzymatic protection systems are not as effective as those situated within the cell, results in an increase in the alkylation phenomena which add to the glycation and glycoxidation products resulting from carbonyl stress.

Accordingly, carbonyl stress, coupled with an extracellular oxidative stress, is at least as significant as the intracellular oxidative stress in the development of aging and the establishment of the tissue alternations that accompany the major pathologies.

The study by the inventors of the phenomena leading to tissue aging has therefore led them to a more extended appreciation of the biochemical mechanisms responsible for such aging and to develop new concepts permitting the definition of new biological targets of complementary action for their more effective control.

Their research has therefore resulted in modification to the structure of polyphenols having antioxidant and free-radical-scavenger properties, such as those which make up plant extracts, in order to provide them with greater abilities to likewise scavenge carbonyl stressors.

It is therefore an object of the invention to provide new compositions of polyphenol derivatives that constitute overactivated polyphenols which both are capable of acting very efficiently on a larger number of biological targets (oxidative and carbonyl stress) and are stabilized.

Another object of the invention is to provide a process for obtaining such polyphenol derivatives from plant extract polyphenols.

In accordance with yet a further aspect, the invention aims to exploit properties of these polyphenol compositions of phloroglucinol type in cosmetology, dietetics, and therapeutics.

The polyphenol derivative compositions of the invention are characterized in that said polyphenols comprise monomers, oligomers or polymers of units conforming to the formula (I):

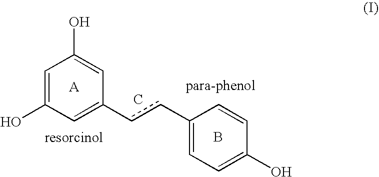

These units are characterized by the simultaneous presence of a resorcinol nucleus (nucleus A) and of a para-phenol nucleus (nucleus B), which are joined to one another by a carbon linkage such as C. In the simplest case, the two nuclei A and B are merged and the segment C is absent, corresponding to the case of phloroglucinol of formula (II):

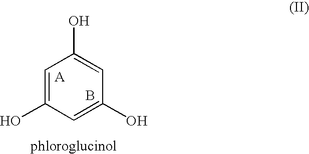

The nuclei A and B of these units are most often separate, and the segment C is composed of 2 carbons, which in one alternative are sp2 hybridized and form a vinyl: this is the case for resveratrol of formula (III):

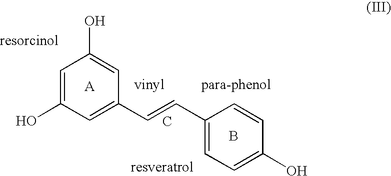

Segment C may also be composed of sp3 hybridized carbons and may serve, in particular, as a point of attachment between the monomers, for forming polymers.

Said derivatives are overactivated, with regard to their nucleophilic power, by alkylation of at least one phenolic function of each unit, and are stabilized by esterification, with mixtures of predominantly unsaturated fatty acids (UFA), of all of the others which have remained free.

Generally speaking, the specific substitutions of the derivatives in the compositions of the invention lead to a modulation of their activity and enable them, at the same time and specifically, to inhibit the principal mechanisms involved in the primary pathologies and the aging as set out above.

Advantageously, the number of —O-alkyl groups per molecule is not equal to the number of hydroxyls present on average per molecule, and preferably is 1 or 2.

The alkyl group or groups are more particularly groups having an electron donor effect: methyls, isopropyls or tert-butyls, for maximum boosting of the nucleophilicity of the aromatic nuclei and, consequently, of their capacity to scavenge carbonyl stressors.

Effective stabilization is obtained by formation of UFA esters between the phenolic functions that have remained free and the fatty acids from vegetable oils containing predominantly unsaturated fatty acids (UFA). The oils are selected for their favorable effect on health. Advantageously, the active substances obtained then contain proportions of unsaturated fatty acids that are identical with those of the oils from which they originate.

Said esters preferably comprise the mixtures of acyl radicals R from the fatty acids of olive oil (Olea europea) or grapeseed oil (Vitis vinifera).

The radicals in question are more especially radicals R of saturated fatty acids (SFA=stearic acid; 7-8%), of monounsaturated fatty acids (MUFA=oleic acid; 55-75%), and of essential polyunsaturated fatty acids (PUFA; 15-18%): diunsaturated (linoleic acids) and triunsaturated (linolenic acids) of the ω-6 and ω-3 series, which are present in the derivatives of the invention in proportions identical to those of the oils which produce a maximum benefit for health, according to the data obtained from epidemiology.

This stabilization makes it possible, furthermore, to protect the overactivated stilbene polyphenols from certain premature destruction (oxidation in the air or in the light), while giving them a lipophilic character in order to enhance their chances of being absorbed.

Advantageously, however, this stabilization is temporary, and is no longer effective when the derivatives are put in place to act, so as to restore to them all of their antioxidant power. The stabilization must therefore be reversible by the simple action of the biological systems to which the stabilizing groups are then exposed, and especially enzymes such as lipases, esterases or proteases.

More specifically, the invention relates to compositions characterized in that said derivatives conform to the formula (IV):

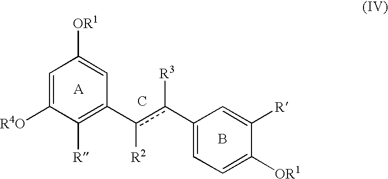

in which
R¹ is an alkyl radical, or an acyl radical of a fatty acid of a vegetable oil, represented by R as defined above,
R² is a hydrogen or the junction point at R″ or to R² of another unit,
R³ is a hydrogen or the junction point at R″ or at R⁴ of another unit,
R⁴ is an alkyl radical, or an acyl radical of a fatty acid of a vegetable oil, represented by R as defined above, or the junction point at R³ of another unit,
R″ represents H or the junction point at R² or at R³ of another unit,
R′ is a hydrogen or an O-acyl radical of a fatty acid of a vegetable oil, represented by R as defined above

and the diastereoisomers and regioisomers of these moieties.

As an example, it is possible to give the dimer (epsilon-viniferin) and trimer (miyabenol C), of formulae (V) and (VI):

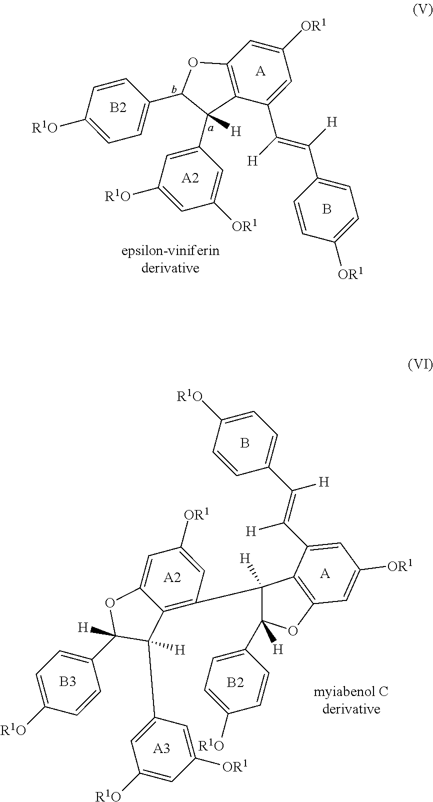

According to one preferred embodiment of the invention, the derivatives defined above correspond to plant extract derivatives which have been alkylated and then stabilized. They therefore have the structures of the polyphenols present as a mixture in these plant extracts.

Accordingly, said plant extracts are essentially composed of derivatives of resveratrol, the latter conforming to the formula (III):

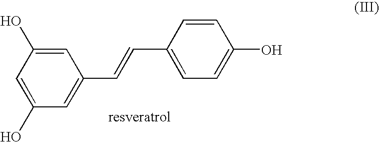

In a first group of this class, the extracts are more particularly vine extracts.

The invention relates especially to derivatives of extracts of vine shoots and/or stems (Vitis vinifera).

The invention relates accordingly to compositions of polyphenol derivatives from vine shoot extracts, these extracts comprising large amounts of polyphenol derivatives which constitute, as indicated earlier on above, vinylogous equivalents of phloroglucinol. These are, especially, polyphenols of formulae III, VII, VIII, IX, and X below, corresponding, respectively, to resveratrol, piceatannol, epsilon-viniferin, pallidol, miyabenol C.

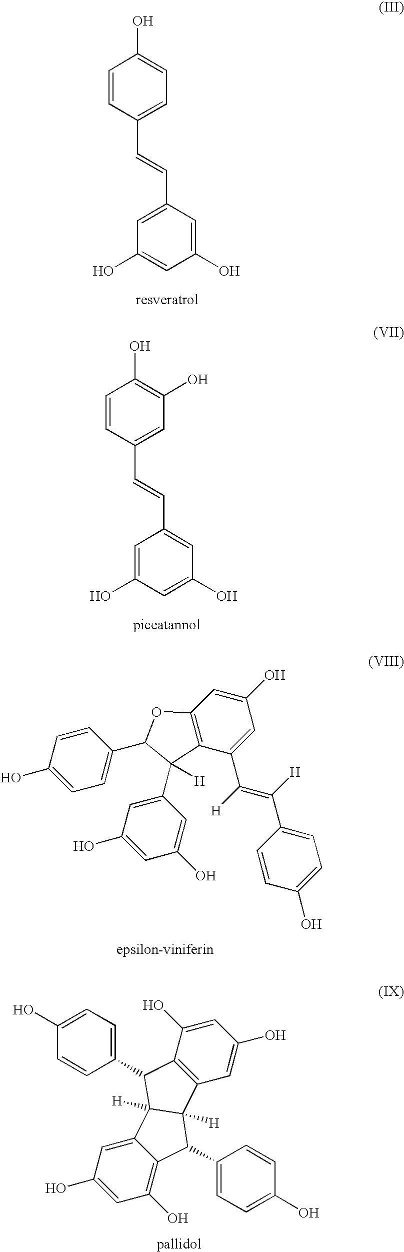

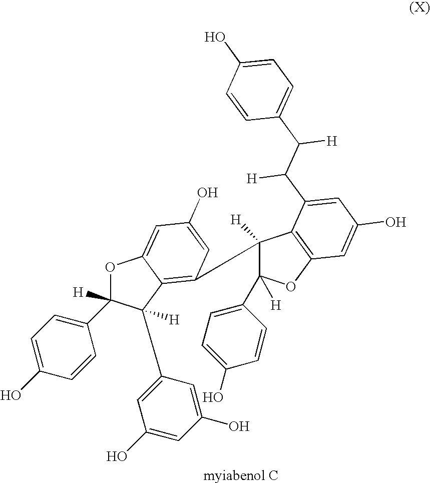

In a second group of said first class, the derivatives are derivatives of Polygonum extracts (Polygonum cuspidatum).

In a third group, the derivatives are derivatives of fruit extracts, such as of mulberry plants (Morus sp).

The compositions of polyphenol derivatives of the invention are advantageously obtained by a process comprising the reaction of the plant extract polyphenol compositions defined above
in a first step, with an alkylating agent under conditions allowing substitution of an alkyl group for the hydrogen of at least 1 phenolic OH group per molecule, preferably of 1 to 2, and
in a second step, with an acylating agent, especially an acid anhydride or acid chloride, under conditions allowing substitution by a mixture of acyl radicals —COR liberated by the acylating agent, R being as defined above, for the hydrogen of the —OH groups which are still free after alkylation.

The alkylation reaction employs reactants which are available commercially, such as halides (iodides, bromides, etc.) or sulfuric esters, in a proportion of one-and-a-half chemical equivalents. They are added slowly to a solution of the polyphenol extract in an aprotic solvent (anhydrous acetone, for example), and in the presence of an inorganic base (potassium carbonate, etc.), which is heated at reflux, with stirring and under an inert atmosphere (nitrogen, argon, ideally).

The alkylation reaction is halted, after cooling, by addition of a dilute acid (hydrochloric acid, for example) until an acid pH is obtained. Stirring is continued for 45 additional minutes, approximately. The reaction mixture is concentrated under vacuum (evaporation of the solvent). The aqueous phase is extracted with an equal volume of immiscible solvent (ethyl acetate, dichloromethane, etc.), which is itself washed with two equivalent volumes of distilled water (until neutrality). This organic phase is dried over anhydrous sodium sulfate and then filtered and evaporated under reduced pressure to leave the residue of the alkylated polyphenols.

The acylating agent is prepared from a vegetable oil by a process comprising:
the saponification of the glycerides of the vegetable oil, followed by an acidification,
activation by dehydration where the acylating agent is an acid anhydride, or by chloridation where it is an acid chloride, although other derivatives imparting the same activating effect may be used (transesterification, enzymatic acylation, as appropriate).

The saponification reaction is performed in aqueous phase in the presence of an alkaline agent such as potassium hydroxide in an at least stoichiometric amount, preferably at the reflux temperature. The solution is then brought to acid pH by addition of inorganic acid, then extracted with an organic solvent so as to isolate the mixture of the free acids formed during the reaction.

The dehydration reaction takes place at reflux, in the presence of a solvent capable of producing an azeotrope with water, so as to allow it to be removed in line with its formation. Toluene, for example, is used, and the water is trapped by a Dean Stark system.

The chloridation reaction is conducted in the presence of a solvent capable of dissolving the free fatty acids. It is catalyzed by Lewis base and carried out by slow addition of the chloridating agent, at a controlled temperature, close to 0° C. When the addition is ended, stirring is continued at the ambient temperature and the reaction mixture is then concentrated by evaporation under vacuum, and the chlorides are purified by distillation.

Advantageously:
the solvent used for the chloridation is dichloromethane or chloroform, for example, provided it is not stabilized by an alcohol,
the chloridating agent is, for example, thionyl chloride or oxalyl chloride,
the catalyst may be dimethylformamide,
the acyl chlorides are purified by distillation under a high vacuum, in a “ball oven” (Kugelrohr).

The acylation reaction is usually carried out in the presence of a solvent which allows solubilization, even partial solubilization, of the alkylated polyphenol compounds resulting from the alkylation reaction described above.

Appropriate solvents are selected from halogen derivatives such as dichloromethane, chloroform or 1,2-dichloroethane, or nitrogen derivatives such as pyridine, or even hexane, depending on the alkylated compounds to be dissolved.

The alkylated polyphenol derivatives, in solution in the selected reaction solvent, and advantageously admixed with a basic catalysis agent (for example, triethylamine or pyridine), are placed under stirring and in an inert atmosphere (argon, nitrogen).

Two equivalents of FA anhydrides or chlorides, as prepared above, are used as acylating agents. They are added dropwise, in solution in the solvent for the reaction, unless that solvent is pyridine alone. Where pyridine is both the solvent and the basic catalyst, an “inverse” addition is operated. This involves the solution of the polyphenol derivatives being added dropwise to the acylpyridinium compounds formed beforehand.

One alternative which may be employed involves adding, with vigorous stirring, a basic aqueous phase (Na₃PO₄, K₃PO₄) to the organic solution (CHCl₃, CH₂Cl₂) of the alkylated polyphenol derivatives and of the acylating agents, thus producing Schotten-Baumann conditions.

Whatever procedure is adopted, the reaction is carried out preferably at ambient temperature, for a time of approximately 7 to 8 hours.

The esterified derivatives thus formed are purified by addition of acidulated water (HCl, qs acid pH), then by a number of washes of the organic phase with distilled water. After drying over sodium sulfate, the solution is filtered and then evaporated to dryness to yield the stabilized and alkylated active flavonoid substances.

The dual-effect active substances of the invention, capable of trapping not only the ROS, irrespective of their intracellular or extracellular origin, but of also trapping the dicarbonyl compounds (antiglycation and anti-AGEs), are of great interest as the most comprehensive and most effective means to date for combating skin aging.

The compositions of the invention are therefore particularly appropriate for the production of cosmetic preparations.

In these preparations, the compositions are combined with vehicles which are appropriate for external use. Advantageously, their fat-soluble nature favors their incorporation into the product forms that are commonly used in cosmetology.

The invention is therefore directed to cosmetic compositions characterized in that they comprise an amount effective for controlling skin aging of one or more compositions of stilbene polyphenol derivatives as defined above in combination with inert vehicles which are appropriate for external use.

These compositions take a form appropriate for topical administration, such as cream, ointment, emulsion, gel, liposomes, lotion.

They contain from 0.5% to 5% of active product, preferably from 2% to 3%.

The invention also relates to a method of preventing skin aging, characterized by the application to the skin, or the ingestion, of one or more cosmetic compositions as defined above.

According to another aspect of great interest, the compositions of the invention can be used in dietetics. By virtue especially of their anti-free-radical and carbonyl-compound-scavenging properties, they ensure better preservation of foods. Moreover, they generally constitute a provider of vitamin factor. They are therefore added with advantage to drinks, as for example to fruit juices, tonic drinks, to dairy products and derivatives such as butter. They can also be used as they are in liquid form, or else as granules or the like, gels or in paste form, incorporated, for example, into confectionery such as fruit gums, candies, chewing gums.

The properties of the compositions of the invention are also advantageously exploited for use as medicaments.

The invention thus also relates to pharmaceutical compositions characterized in that they comprise a therapeutically effective amount of at least one composition as defined above, in combination with a pharmaceutically acceptable vehicle.

These compositions advantageously take a form appropriate for—in particular—oral, topical or parenteral administration.

Accordingly, for oral administration, the compositions take the form more particularly of tablets, gel capsules, solutions or syrups.

For topical administration, the compositions take the form of cream, ointments, gels, patches or lotions.

For parenteral administration, the compositions take the form of a sterile or sterilizable injectable solution.

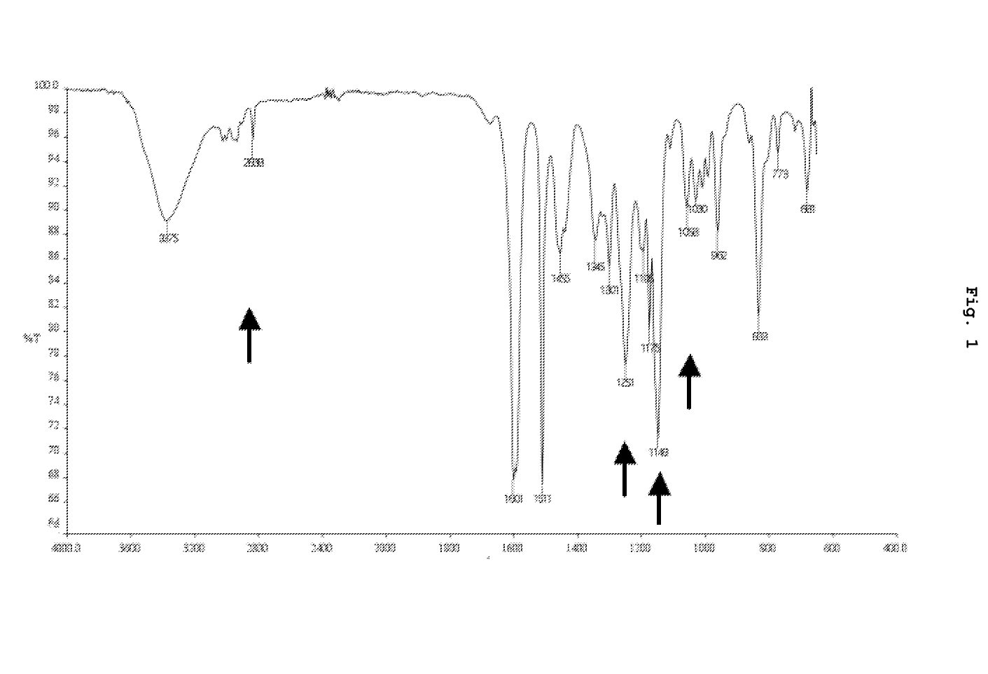

Other characteristics and advantages of the invention are given, by way of illustration, in the examples which follow, in which reference is made to FIGS. 1 to 11, which represent respectively:

FIG. 1: the FT-IR spectrum in ATR mode of resveratrols monoalkylated (methylated) by methyl iodide,

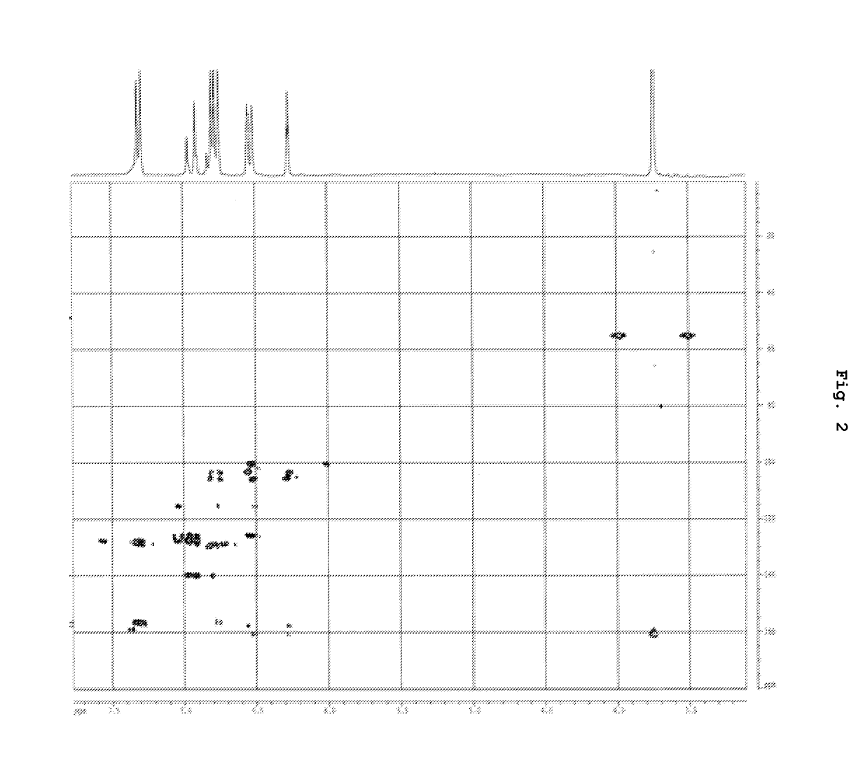

FIG. 2: the ¹H-¹³C HMBC 2D NMR spectrum (500 MHz) of resveratrols monoalkylated (methylated) by methyl iodide,

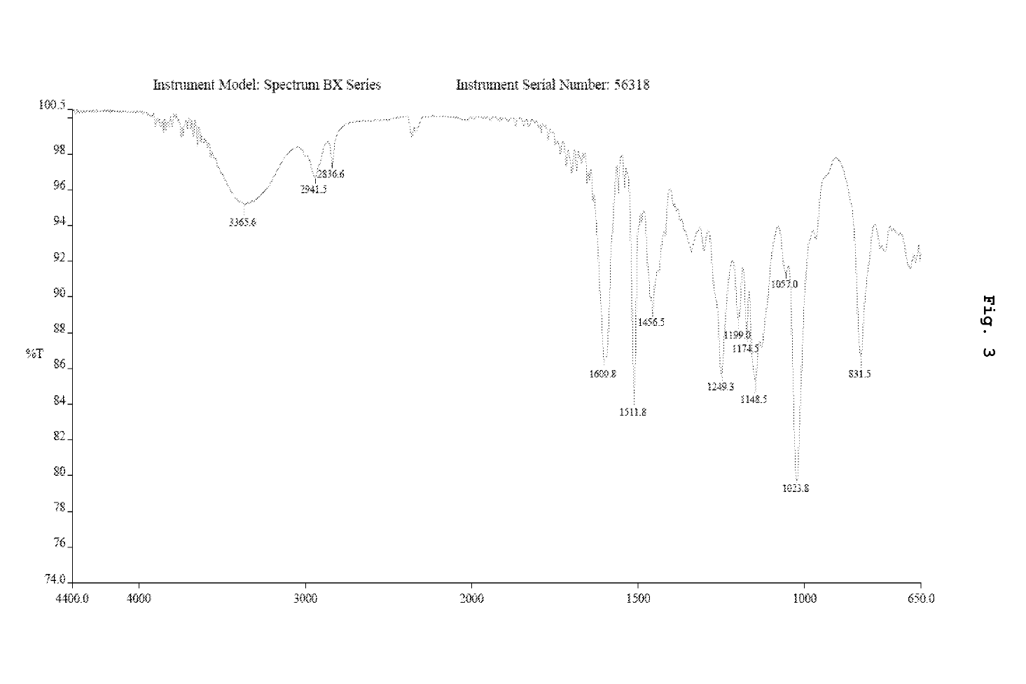

FIG. 3: the FT-IR spectrum of resveratrols monoalkylated (methylated) by DMS,

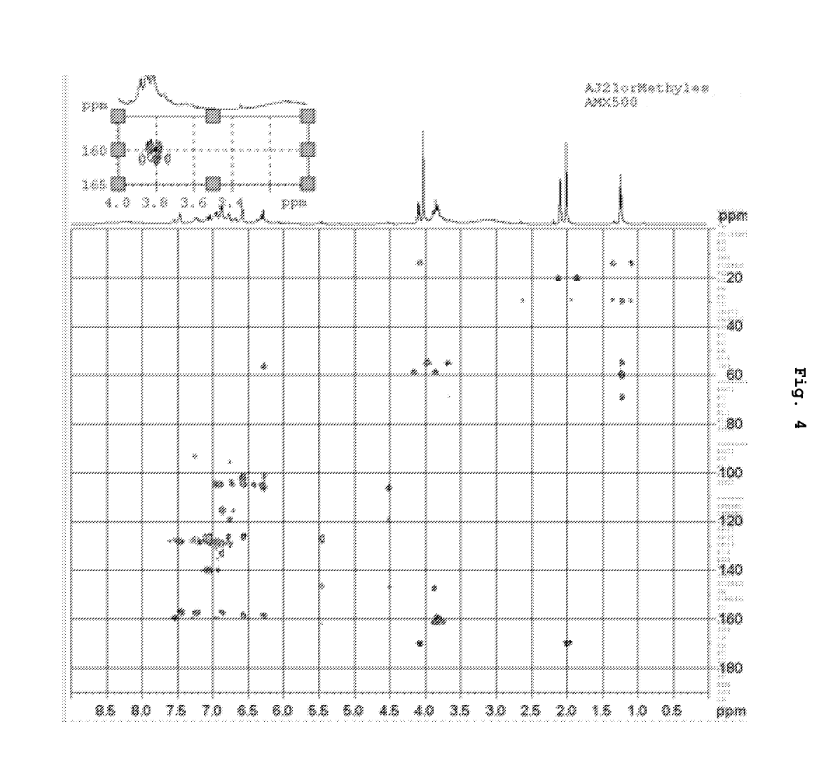

FIG. 4: the ¹H-¹³C HMBC 2D NMR spectrum (500 MHz) of resveratrols monoalkylated (methylated) by methyl iodide,

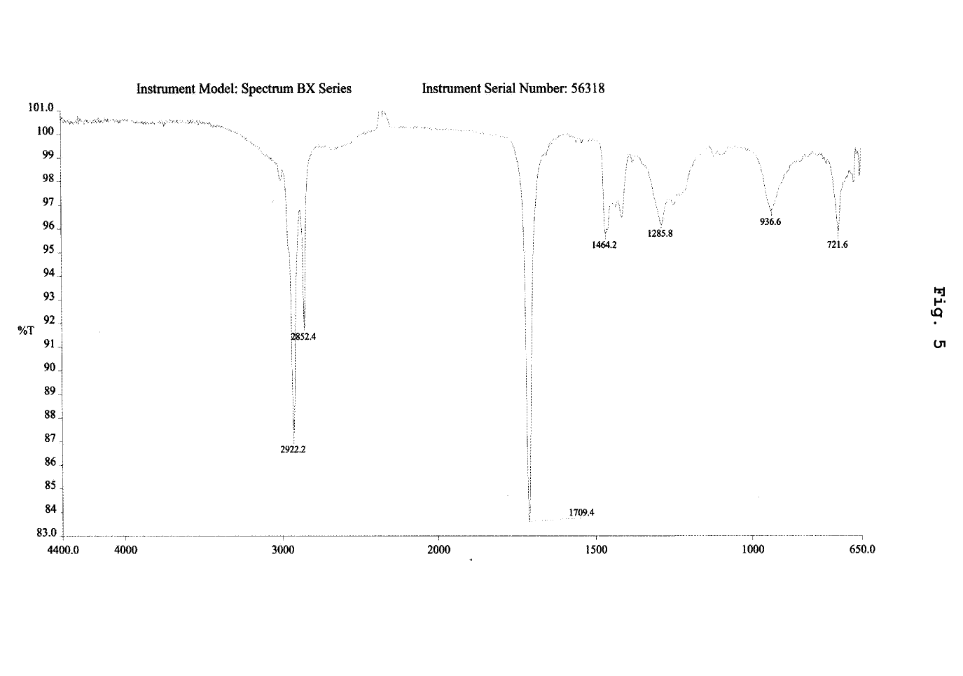

FIG. 5: the FT-IR spectrum of the fatty acids obtained from the saponification of a “virgin” olive oil, in ATR mode,

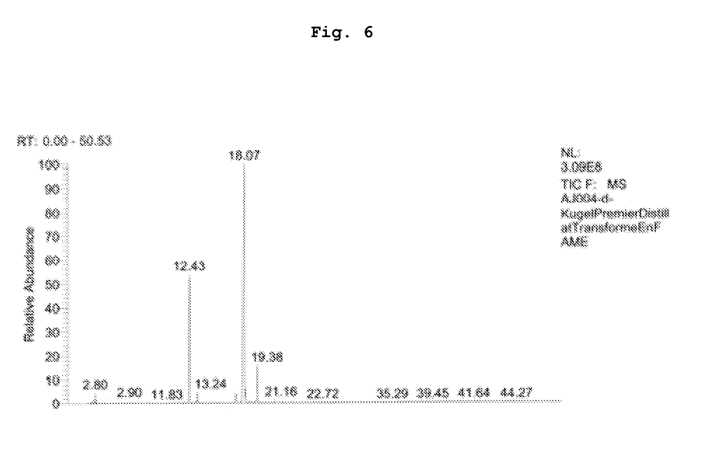

FIG. 6: the gas chromatogram, detected by mass spectrometry (GC-DSQ2), of the methyl esters prepared from olive FA chlorides,

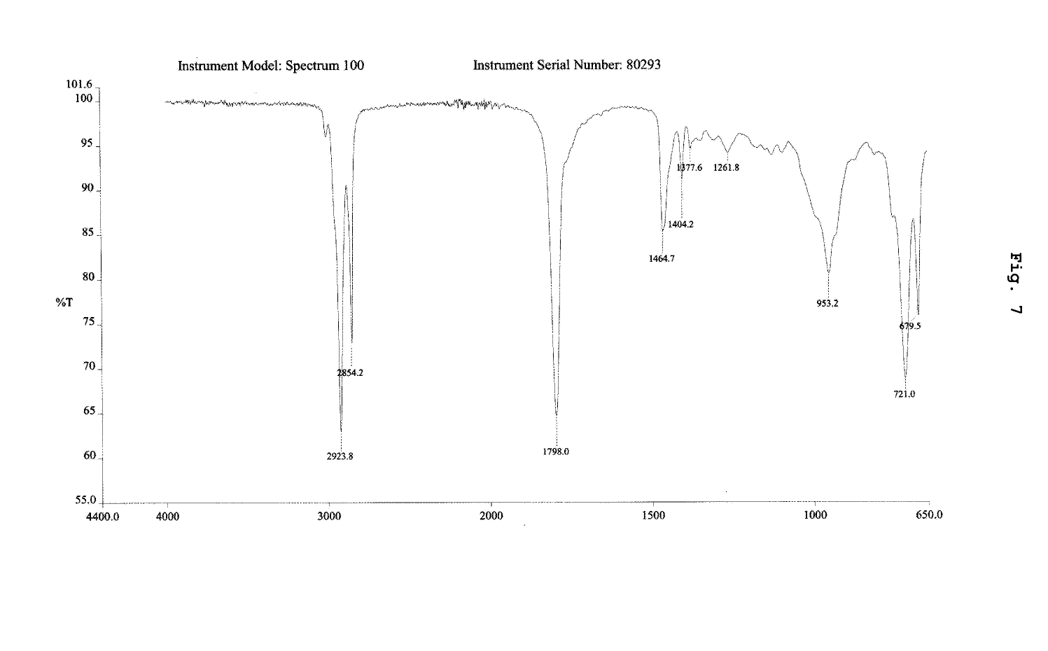

FIG. 7: the FT-IR spectrum of olive FA chlorides (in ATR mode),

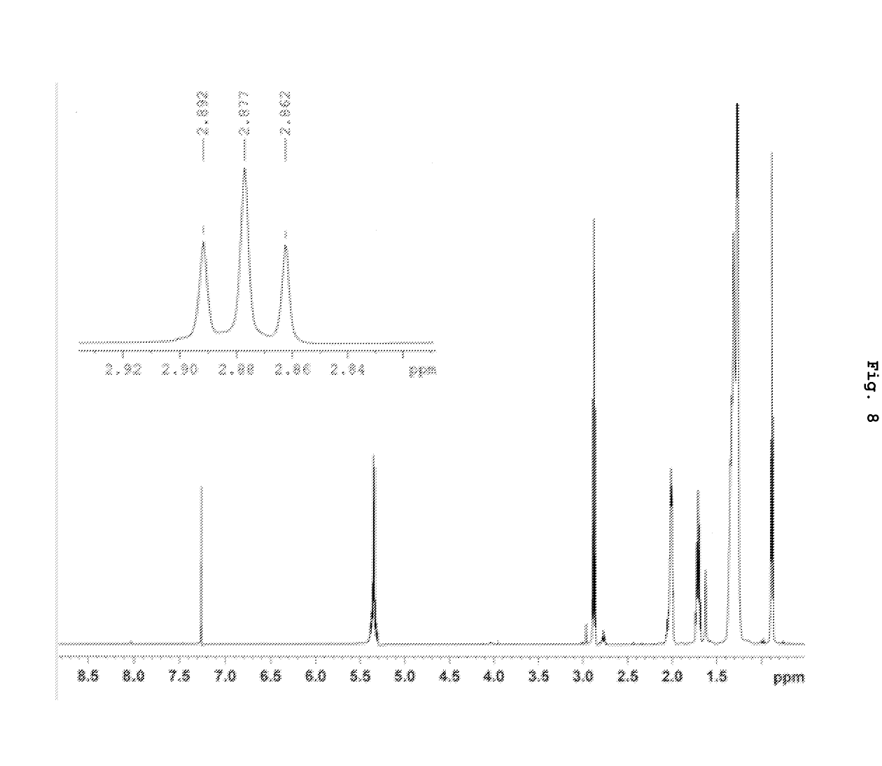

FIG. 8: the proton NMR spectrum at 500 MHz (CDCl₃) of olive FA chlorides,

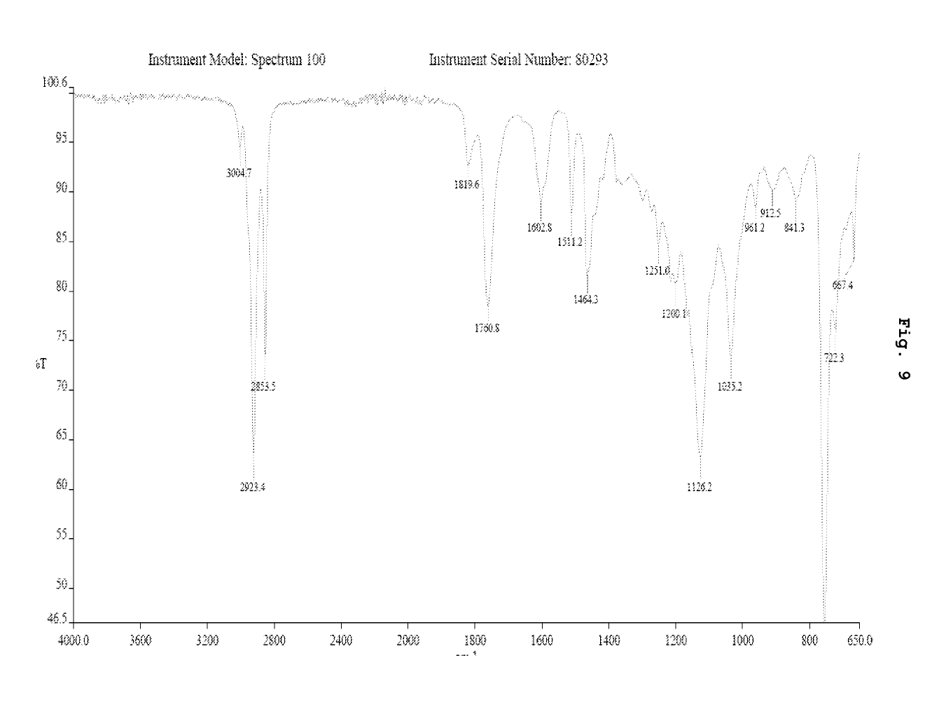

FIG. 9: the FT-IR spectrum of stilbenoid polyphenols from vine shoots, alkylated and stabilized with olive oil FAs,

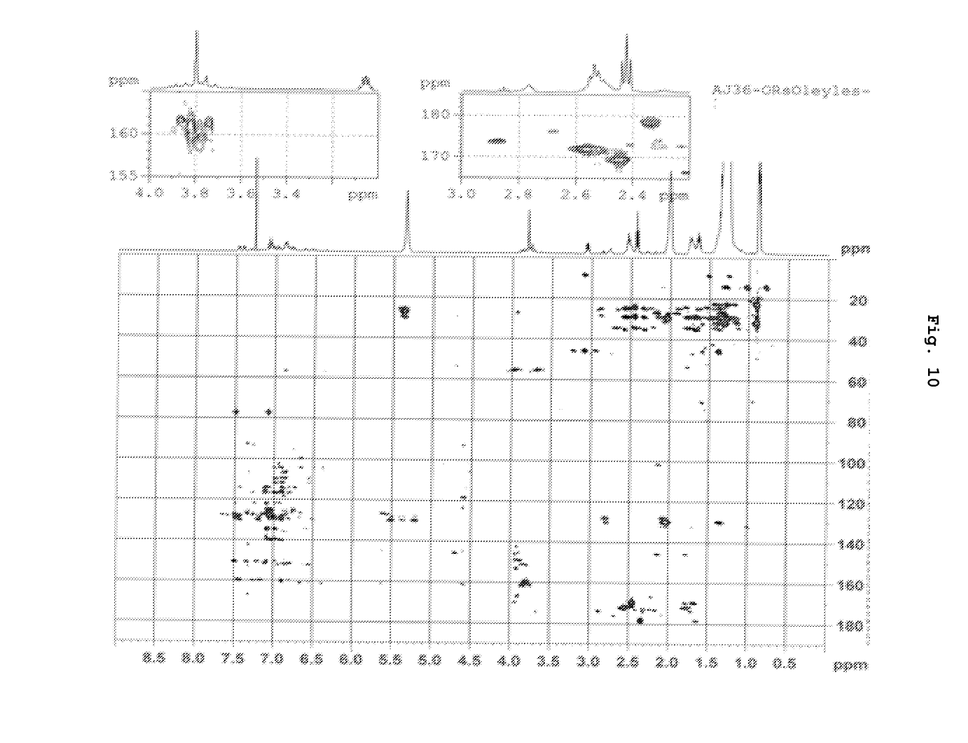

FIG. 10: the ¹H-¹³C HMBC 2D NMR spectrum (500 MHz, CDCl₃) of stilbenoid polyphenols from vine shoots, alkylated and stabilized with olive oil FAs.

### EXAMPLE 1

### O-alkylation of Phloroglucinol

1.560 g of phloroglucinol (12.3 mmol) are dissolved in 20 ml of anhydrous acetone in a double-necked flask with a top-mounted condenser. With stirring under an argon atmosphere, in the presence of 1.685 g (12.3 mmol, 2 chemical eq) of potassium carbonate (K2CO₃), 766 microliters of methyl iodide are added (=1.746 g; d=2.28 g/ml at 25° C.), i.e., 12.3 mmol=1 molar equivalent relative to the resveratrol. The reaction is heated at reflux for 3 hours.

The reaction mixture is filtered on a No. 4 frit to remove the K₂CO₃, and the acetone is evaporated under vacuum. The residue is taken up in 15 ml of ethyl acetate. The organic phase is washed with 2 times 15 ml of distilled water, dried over sodium sulfate, filtered and evaporated to dryness to leave a residue of 1357 mg, which is identified as 5-methoxyresorcinol (crude yield=89%; mw=124) on the basis of its spectral constants: ¹H NMR, acetone-d6, 500 MHz, δ ppm: 5.95 (1H, d); 5.90 (2H, d); 3.65 (3H, s, CH₃). ¹³C NMR, acetone-d6, 125 MHz, δ ppm: 167.2 (s); 164.22 (2 s); 100.61 (d); 98.26 (2 d); 59.6 (quad.).

### EXAMPLE 2

### O-alkylation of Resveratrol

450 mg of resveratrol (1.97 mmol) are dissolved in 10 ml of anhydrous acetone in a double-necked flask with a top-mounted condenser. With stirring under an argon atmosphere, in the presence of 270 mg (1.97 mmol, 2 chemical eq) of potassium carbonate (K₂CO₃), 123 microliters of methyl iodide are added (=280 mg; d=2.28 g/ml at 25° C.), i.e., 1.97 mmol=1 molar equivalent relative to the resveratrol. The reaction is heated at reflux for 3 hours.

The reaction mixture is filtered on a No. 4 frit to remove the K₂CO₃, and the acetone is evaporated under vacuum. The residue is taken up in 15 ml of ethyl acetate. The organic phase is washed with 2 times 15 ml of distilled water, dried over sodium sulfate, filtered and evaporated to dryness to leave a residue of 548 mg (crude yield=91.6%, on the basis of monomethylated resveratrols; mw=304).

FT-IR spectroscopic study of this extract of O-methylated resveratrols establishes the characteristics which are common to all of these methylated derivatives, including the most complex of the plant stilbenoid polyphenol extracts, labeled with arrows in the spectrum (FIG. 1): bands at 2838 (CH) and 1251, 1143, and 1058 cm⁻¹ (ethers).

This mixture of monomethylated resveratrols is characterized in NMR by the correlations, which are indicative of alkylation, between aromatic oxygen-bearing carbons in the resveratrol (δ=160 ppm) and the protons of methyl ethers (δ=3.8 ppm). The HMBC 2D NMR spectrum (FIG. 2) shows correlations between the oxygen-bearing aromatic carbons (from 155 to 162 ppm) and the protons of methyl ethers, which resonate at a frequency centered on 3.8 ppm.

### EXAMPLE 3

### Step of O-alkylation of Stilbene Polyphenols

Operation takes place on 10.08 g (44 mmol) of stilbenoid polyphenol extract as obtained by the process of patent FR 2 766176, which are dissolved in 50 ml of acetone. 12.25 g of activated K₂CO₃ (88 mmol=4 chemical eq) and then 3.15 ml (33 mmol, 1.5 chemical eq) of the alkylating agent, in this case dimethyl sulfate (DMS), are added with stirring under an argon atmosphere.

The calculation of the chemical equivalents is made on the basis of an “average” of 3 hydroxyl residues per unit of resveratrol. Thus it is considered that each portion of 228 g of extract corresponds to “1 mol of resveratrol”, and possesses three phenolic functions, of which only one is to be converted to alkyl ether. The chemical equivalent of the alkylating reactant is therefore equal to a third of the number of moles of resveratrol extract employed, on the basis that the molecular weight is 228.

The clear solution obtained is heated at reflux for 7 hours, and the reaction is cooled. Following addition of a dilute hydrochloric acid solution, until an acid pH is obtained (220 ml), stirring is continued for 45 minutes more. The reaction mixture is concentrated under vacuum (evaporation of the acetone). The residual aqueous phase is extracted with an equal volume of ethyl acetate, which is washed with two times 200 ml of distilled water (until the washing water is neutral). This organic phase is then dried over anhydrous sodium sulfate, filtered, and evaporated under reduced pressure, to leave the residue of the alkylated stilbenoid polyphenols (9.923 g; crude yield=93.2%, average mw=242).

In the preferred case where each molecule of the initial extract undergoes a single methylation per stilbenoid unit (“resveratrol”), a mixture of the various possible regioisomers and stereoisomers is obtained, such as the monomers and dimers featured below.

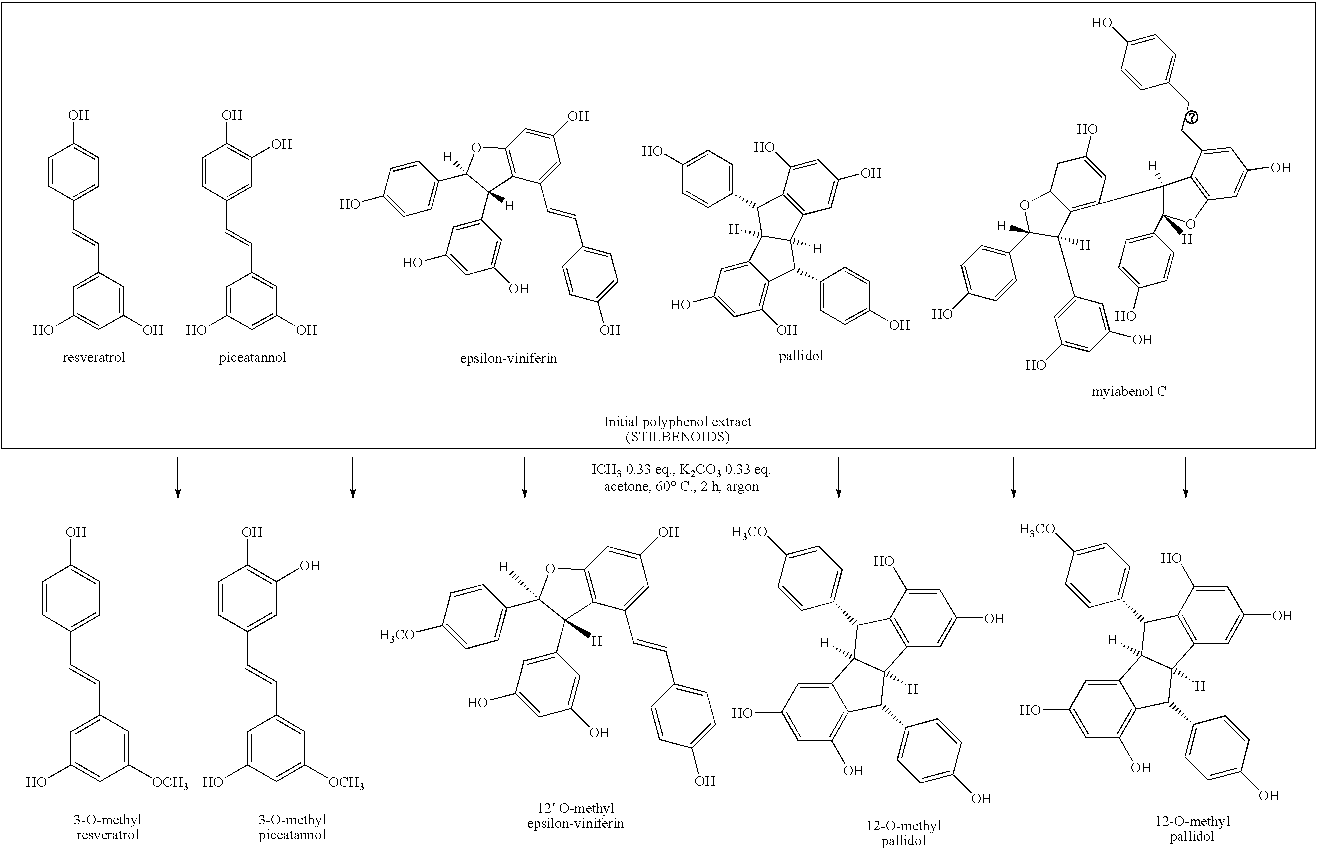

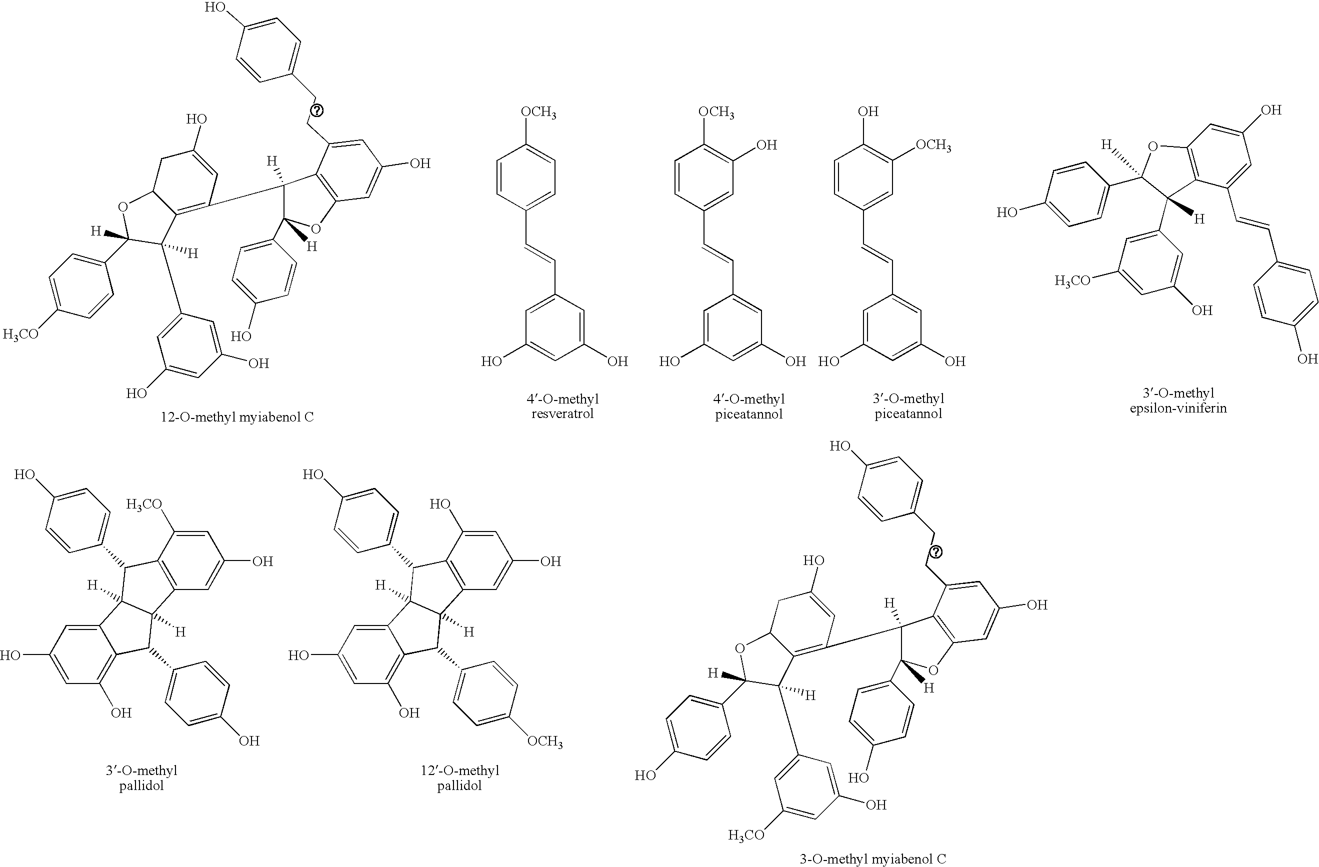

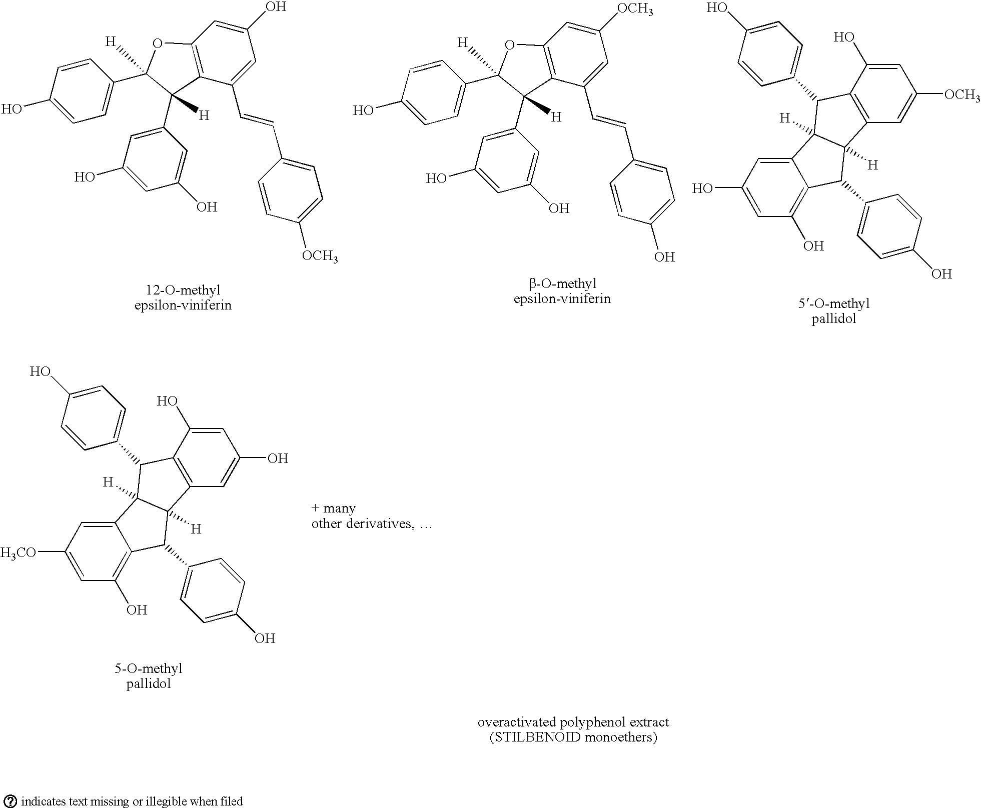

### Examples of Stilbenoid Polyphenols Activated against Carbonyl Stresses

Generally speaking, however, the different phenolic functions of each of the molecules react with different kinetics. For resveratrol, for example, the proportions between the various position isomers are as follows:

| | |
|---|---|
| resveratrol | 6% |
| 3-O-methyl-resveratrol | 8% |
| 4′-O-methyl-resveratrol | 13% |
| 3,4′-di-O-methyl-resveratrol and | 23% |
| 3,5′-di-O-methyl-resveratrol | |
| 3,4′,5-tri-O-methyl-resveratrol | 13% |

The result of this is a great diversification of the overactivated active stilbenoid substances which are composed of monomethylated derivatives of the above figure, but they are nevertheless accompanied, to a minority degree, by possible di- and tri-methylated isomers.

As for the preceding example, the alkylated (methylated) structures of these stilbenoid compounds are deduced from the analysis of their various spectra:
The presence of phenolic methyl ethers is manifested in IR (FIG. 3), in particular, by the appearance of absorption bands between 2974 and 2836 cm⁻¹ which are characteristic of methyl C—H (elongation) and, between 1040 and 1235 cm⁻¹, those which are characteristic of ether (C—O) functions.
The HMBC 2D NMR spectrum (FIG. 4) shows correlations between oxygen-bearing aromatic carbons (from 155 to 160 ppm) and the protons of methyl ethers, which resonate at a frequency centered on 3.8 ppm.

### EXAMPLE 4

### Preparation of Acylating Agents

Step 1: Saponification of Olive Oil:

50.46 g of “virgin” olive oil (57 mmol, =“171 eq”), placed in a round-bottom flask equipped with a condenser, are admixed with 16.08 g of potassium hydroxide (285 mmol, 1.67 eq) in solution in 2.5 ml of ethanol and 50 ml of water. The reaction is taken to reflux for 5 hours. It is stirred for a further 14 h, at ambient temperature.

After the resulting solution has been extended with 300 ml of water, tenth-concentration (3.7%; w/v) hydrochloric acid is added until an acid pH is obtained in the aqueous phase (approximately 250 ml). The contents of the round-bottom flask, which comprises a pasty “insoluble” product at the surface, are then transferred to a separating flask and extracted with 700 ml of hexane. The organic phase is separated off and then washed with 2 times 300 ml of distilled water (to give a neutral pH of this aqueous phase).

The organic phase is dried over sodium sulfate, filtered on a No. 4 frit, and then evaporated to yield a residue of 42.9 g (crude yield=88.8%).

The infrared spectrum recorded in ATR mode with Fourier transform (FIG. 5) shows a band which is characteristic of free organic acids at 1709 cm⁻¹, along with the disappearance of the ester bands of the starting oil.

Step 2: Activation of Fatty Acids Obtained from the Saponification of Olive Oil by Formation of Chlorides:

The solution of 41.5 g of free fatty acids (147.1 mmol) obtained from step 1 in 232 ml of chloroform (stabilized on amylene) is stirred under an argon atmosphere in a round-bottom flask which is cooled by an ice bath. Using a dropping funnel, 13.8 ml of oxalyl chloride (162 mM=1.1 eq) are introduced dropwise over a period of 30 minutes. 1 ml of dimethylformamide (DMF) is introduced, and stirring is continued over the ice bath for 5 minutes. Concentration of the reaction mixture under reduced pressure (chloroform and oxalyl chloride in excess) then gives 44.3 g of an oily residue with a slight yellow coloration (crude yield=100%).

By distillation in a ball oven (kugelrohr) under a high vacuum (2 mmHg), this residue is decolorized (colorless liquid), by collecting the fractions which distill at from 178 to 195° C.

In order to analyze the composition of the mixture of fatty acid chlorides obtained, a few microliters of distillate are exposed to methanol. The total mixture is then injected into a gas chromatograph equipped with a “FAME” (fatty acid methyl ester) column and an online mass detector (DSQ-II). In the chromatogram shown in FIG. 6, the peak at 17.8 min corresponds to the stearate (M+·=298), that at 18.07 min to the oleate (M+·=296), that at 18.08 min to a linoleate (M+·=294), and that at 19.38 min to the linolenate (M+·=292). Their relative intensities are a good indication of their respective proportions.

The FT-IR (FIG. 7) and proton NMR (FIG. 8) spectra are in perfect agreement with the exclusive formation of these chlorides:
A band at 1798 cm⁻¹, characteristic of acyl chlorides.
The protons alpha to the carbonyl (t, J=7.5 Hz) exhibit a chemical shift at 2.9 ppm, which is characteristic of the conversion of carboxyls to acid chlorides.

### EXAMPLE 5

### Esterification of O-alkylated Resveratrol Oligomers

8.4 g (35 mmol) of O-alkylated resveratrol oligomers according to example 3 are suspended in 106 ml of hexane admixed with 9.3 ml of triethylamine (70 mmol), and are stirred under an argon atmosphere. 10.65 g of the chlorides prepared in example 4, diluted in 45 ml of hexane (35.1 mmol, 1 eq) are added dropwise.

The reaction is left for a further 6 hours with stirring at ambient temperature, before being placed in a separating funnel and washed with 100 ml of tenth-concentration hydrochloric acid, then 90 ml of a 10% (w/v) NaHCO₃ solution in water, and finally with distilled water until neutrality (two times 90 ml). The organic phase is dried over sodium sulfate, filtered, then evaporated to dryness, under reduced pressure. It leaves a residue of 19.21 g of stabilized and alkylated vine shoot active stilbenoid substances (=24.64 mmol; crude yield=70.6%, average mw=774).

With the aim of obtaining means of identifying these active substances, the whole product is then subjected to spectral measurements:
The Fourier-transform infrared spectrum acquired in ATR mode (FIG. 9) shows the appearance of an intense band at 1764 cm⁻¹, which is characteristic of the carboxyls of phenolic esters, simultaneous with the disappearance of the broad band centered on 3350 cm⁻¹, which corresponded to the free phenolic hydroxyls.
The long-distance ¹H-¹³C heteronuclear two-dimensional NMR spectrum at 500 MHz (FIG. 10), obtained in inverse mode (HMBC), clearly shows the correlations which are in perfect agreement with the diversified structures of stilbenoid polyphenols which are alkylated (methyl ethers of aromatic oxygens) and esterified (predominantly unsaturated fatty acid esters, in statistical mixture as resulting from the olive oil used for preparing the acylating agents).

In the preferred case in which each molecule of the initial extract has undergone only one methylation per stilbenoid unit (“resveratrol”), and in which the residual phenolic functions are all acylated by the olive oil FA mixture, a mixture is obtained of the various possible regioisomers and stereoisomers of monomers and dimers that are featured below:

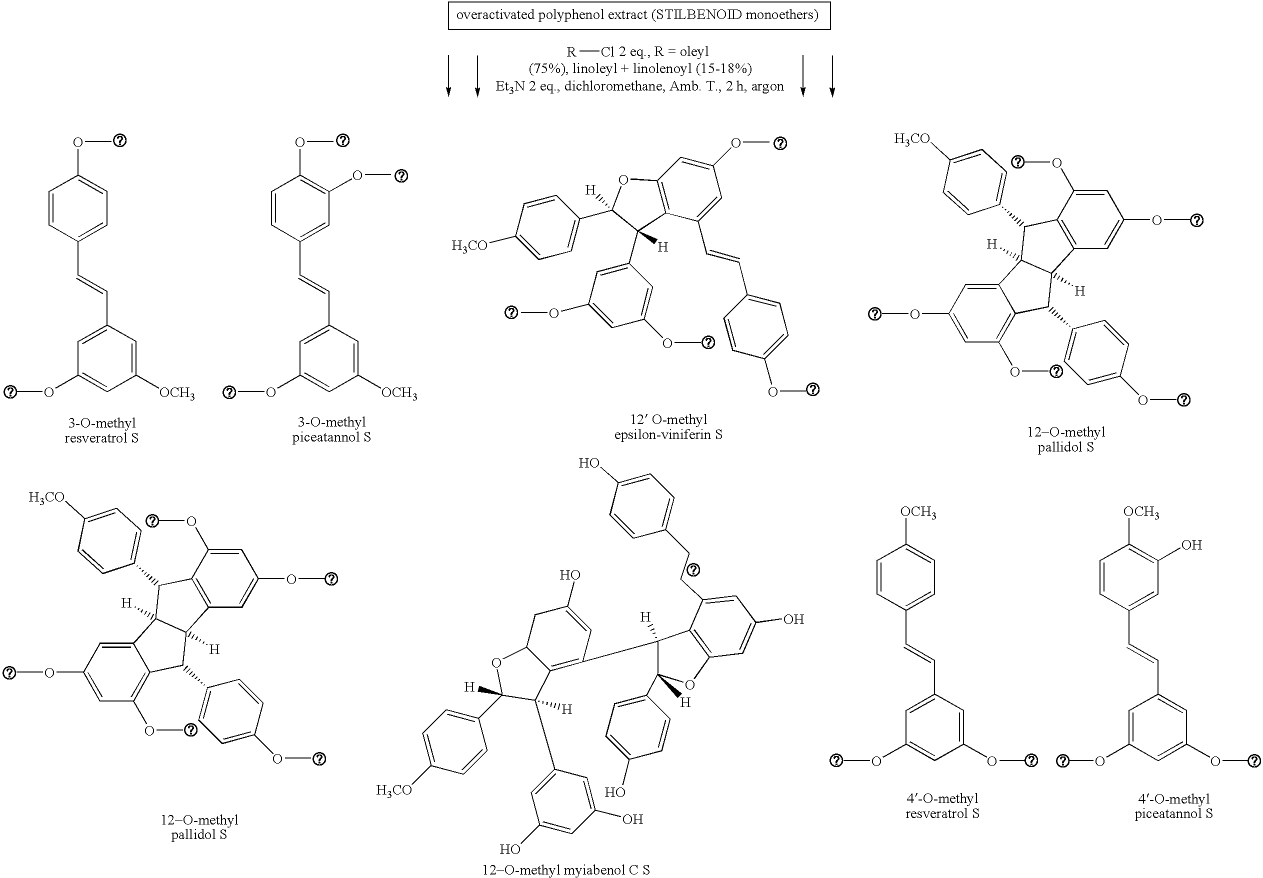

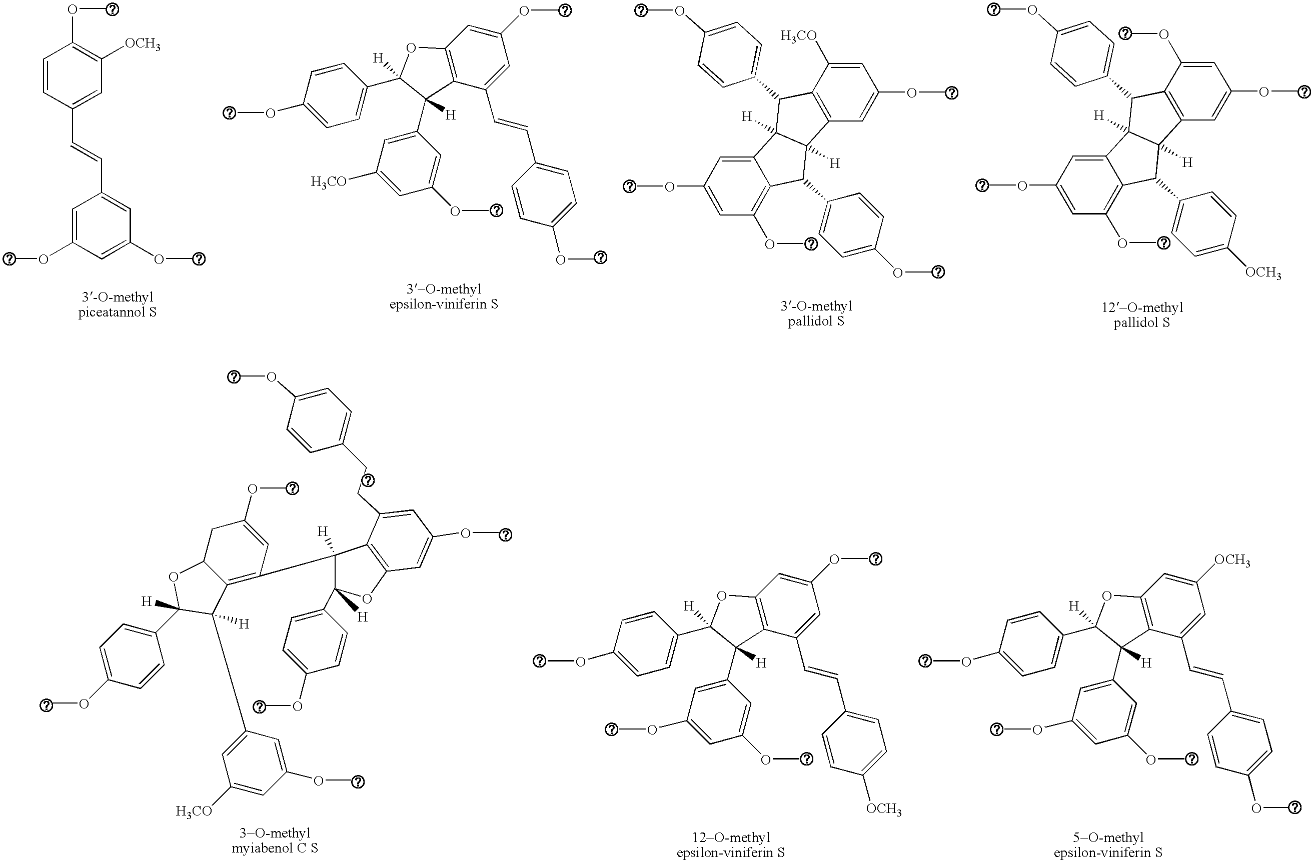

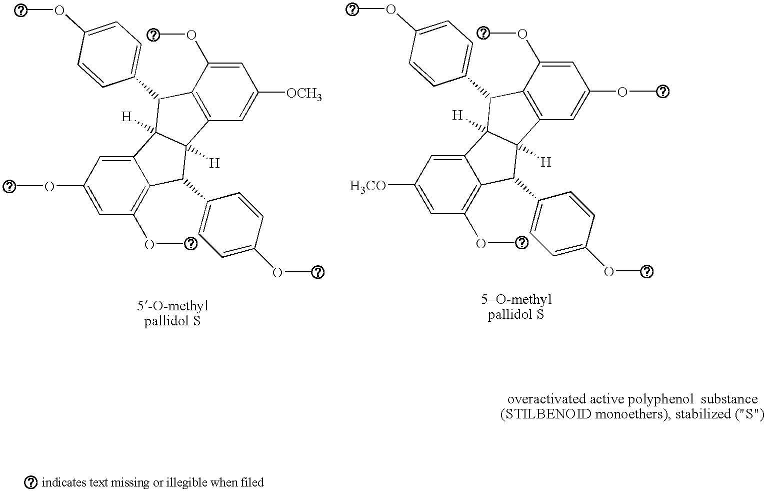

### EXAMPLE 5

### Cosmetic Formulations

FORMULA A

| PHASES | STARTING MATERIALS | % |
|---|---|---|
| 101 | Water | 80.8000 |
| 102 | Tetrasodium EDTA | 0.0500 |
| 103 | Glycerol | 5.0000 |
| 104 | Carbomer | 0.3500 |
| 201 | Wheat cetearyl glycosides | 0.7500 |
| 202 | Barley cetearyl glycosides | 1.7500 |
| 203 | Cetearyl alcohol | 2.5000 |
| 204 | Composition of the invention | 0.05 to 1 |
| 205 | Butyrospermum parkii butter | 2.5000 |
| 206 | Tocopheryl acetate | 0.5000 |
| 207 | Grapeseed oil (Vitis vinifera) | 3.0000 |
| 208 | Cetyl alcohol | 1.0000 |
| 209 | Potassium cetyl phosphate | 1.0000 |
| 301 | Preservatives | 0.6000 |
| 401 | Fragrance | 0.2000 |
| 501 | Sodium hydroxide qs pH 6.00 | |

FORMULA B

| PHASES | STARTING MATERIALS | % |
|---|---|---|
| 101 | Water | 79.40000 |
| 102 | Tetrasodium EDTA | 0.05000 |
| 103 | Citric acid qs final pH 5.5 | 0.15000 |
| 201 | Xanthan gum | 0.30000 |
| 202 | Butylene glycol | 5.00000 |
| 301 | Ceteareth-20 | 1.50000 |
| 302 | Glyceryl stearate | 2.00000 |
| 303 | Composition of the invention | 0.05 to 1 |
| 304 | Butyrospermum parkii butter | 1.00000 |
| 305 | Hexyl laurate | 4.00000 |
| 306 | Dimethicone | 3.00000 |
| 307 | Squalane | 2.00000 |
| 308 | Tocopheryl acetate | 0.50000 |
| 401 | Preservatives | 0.60000 |
| 501 | Fragrance | 0.50000 |

1. Compositions of polyphenol derivatives, characterized in that said polyphenol derivatives comprise monomers, oligomers or polymers of units conforming to the formula (I): 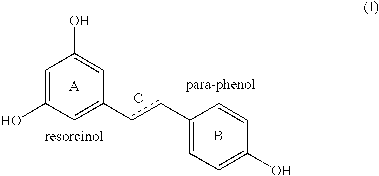
these units being characterized by the simultaneous presence of a resorcinol nucleus (nucleus A) and of a para-phenol nucleus (nucleus B), which are joined to one another by a carbon linkage C, said derivatives being overactivated, with regard to their nucleophilic power, by alkylation of at least one phenolic function of each unit, and being stabilized by esterification with mixtures of predominantly unsaturated fatty acids (UFA) of all of the other phenolic functions.

2. The compositions according to claim 1, characterized in that in said units the nuclei A and B are merged and the segment C is absent, as in phloroglucinol of formula (II):

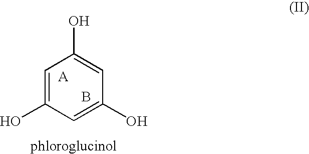

3. The compositions according to claim 1, characterized in that the nuclei A and B in said units are separate, and the segment C is composed of 2 carbons which are sp2 hybridized and form a vinyl, as in resveratrol of formula (Ill): 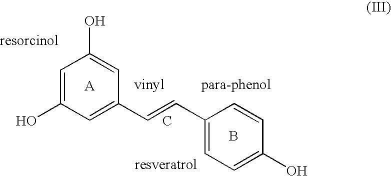
or segment C is composed of sp3 hybridized carbons and serves as a point of attachment between the monomers for forming the oligomers and polymers.

4. The compositions according to claim 1, characterized in that the number of —O-alkyl groups per unit is not equal to the number of hydroxyls present on average per constituent unit.

5. The compositions according to claim 4, characterized in that the number of hydroxyls present on average per unit is 1 or 2.

6. The compositions according to claim 1, characterized in that the alkyl group or groups are methyl, isopropyl or tert-butyl groups.

7. The compositions according to claim 1, characterized in that said esters are fatty acid esters of vegetable oils.

8. The compositions according to claim 7, characterized in that these esters comprise the acyl radicals R corresponding to saturated fatty acids, such as stearic acid, to monounsaturated fatty acids, such as oleic acid, and to essential polyunsaturated fatty acids, such as linoleic and linolenic acids.

9. The compositions according to claim 7, characterized in that the vegetable oils are selected from olive oil or grapeseed oil.

10. The compositions according to claim 1, characterized in that said derivatives conform to the formula (IV): 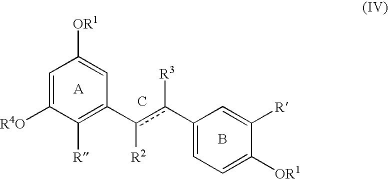
in which
R¹ is an alkyl radical, or an acyl radical of a fatty acid of a vegetable oil, represented by R,
R² is a hydrogen or the junction point at R″ or to R² of another unit,
R³ is a hydrogen or the junction point at R″ or at R⁴ of another unit,
R⁴ is an alkyl radical, or an acyl radical of a fatty acid of a vegetable oil, represented by R as defined in claim 8, or the junction point at R³ of another unit,
R″ represents H or the junction point at R² or at R³ of another unit,
R′ is a hydrogen or an O-acyl radical of a fatty acid of a vegetable oil, represented by R as defined above
and the diastereoisomers and regioisomers of these moieties.

11. The compositions according to claim 10, characterized in that said derivatives correspond to the dimers and trimers of formula V and VI respectively:

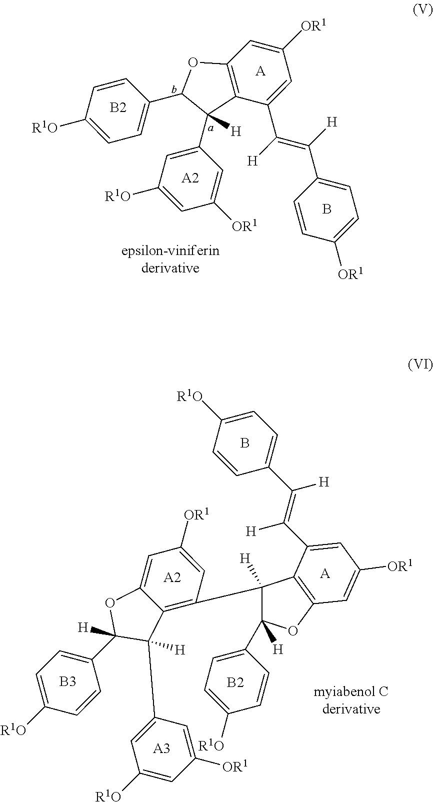

12. The compositions according to claim 1, characterized in that said derivatives correspond to stabilized and alkylated derivatives of plant extracts.

13. The compositions according to claim 12, characterized in that said plant extracts are vine extracts.

14. The compositions according to claim 13, characterized in that said vine extracts are obtained from vine shoots and/or stems.

15. The compositions according to claim 14, characterized in that the constituents in question are derivatives of vine shoot extracts, these extracts comprising polyphenol derivatives which constitute vinylogues of phloroglucinol, especially resveratrol, piceatannol, epsilon-viniferin, pallidol, miyabenol C, corresponding respectively to the formulae III, VII, VIII, IX, and X below:

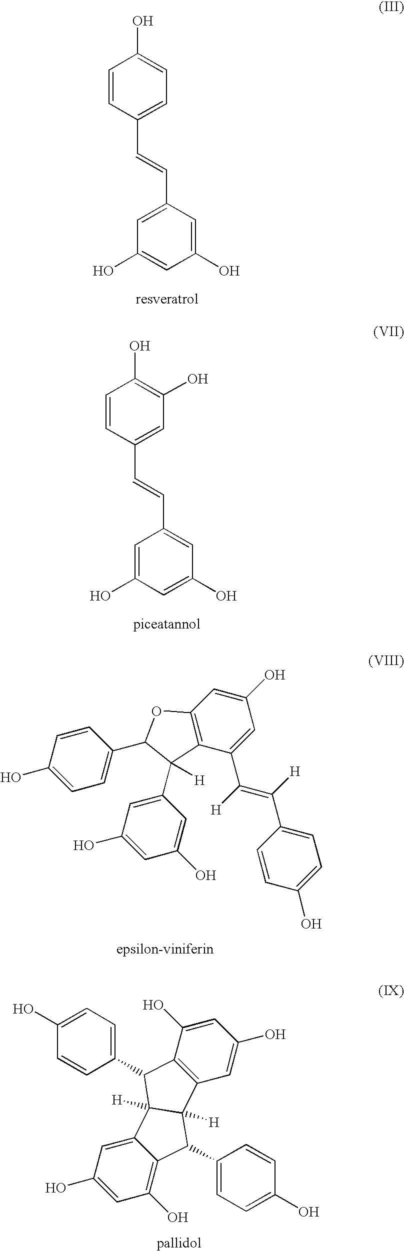

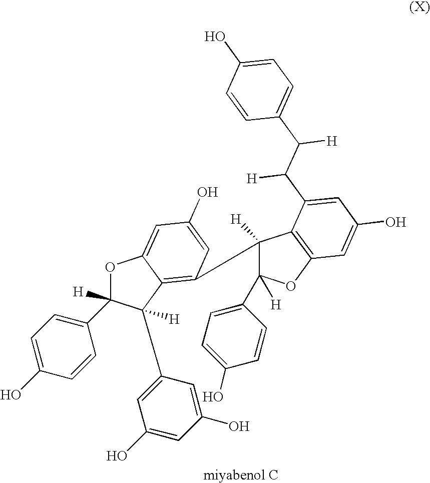

16. The compositions according to claim 12, characterized in that said plant extracts are Polygonum extracts.

17. The compositions according to claim 12, characterized in that said plant extracts are fruit extracts, from mulberry plants, for example.

18. A process for preparing compositions according to claim 1, characterized in that the plant extract polyphenol compositions defined above are reacted
in a first step, with an alkylating agent under conditions allowing substitution of an alkyl group for the hydrogen of at least 1 phenolic OH group per constituent monomeric unit of each molecule, preferably of 1 to 2, and
in a second step, with an acylating agent, especially an acid anhydride or acid chloride, under conditions allowing substitution by a mixture of acyl radicals —COR liberated by the acylating agent, for the hydrogen of the —OH groups which are still free after alkylation.

19. The process according to claim 18, characterized in that the acylating agent is obtained from a vegetable oil by a process comprising:
the saponification of the glycerides of the vegetable oil, followed by an acidification,
activation by dehydration where the acylating agent is an acid anhydride, or by chloridation where it is an acid chloride.

20. Cosmetic compositions characterized in that they comprise an amount effective for combating skin aging of one or more compositions of polyphenol derivatives according to claim 1, in combination with inert vehicles appropriate for external use.

21. The compositions according to claim 20, characterized in that they take a form appropriate for topical administration, such as cream, ointment, emulsion, gel, liposomes, lotion.

22. The compositions according to claim 20, characterized in that they contain from 0.5% to 5% of active product, preferably from 2% to 3%.

23. The application of the compositions according to claim 1, in dietetics.

24. The application according to claim 23, characterized in that said compositions are added to drinks, as for example to fruit juices, tonic drinks, to dairy products and derivatives such as butter, in liquid form, or else as granules or the like, gels, or in paste form, incorporated, for example, into confectionery such as fruit gums, candy, chewing gums.

25. The compositions according to claim 1, for use as medicaments.

26. Pharmaceutical compositions characterized in that they comprise a therapeutically effective amount of at least one composition according to claim 1, in combination with a pharmaceutically acceptable vehicle.

27. The compositions according to claim 25, characterized in that they take a form appropriate for administration by oral, topical or parenteral administration.

28. The compositions according to claim 27, characterized in that they take a form for oral administration, such as solution, syrup, tablet, gel capsule.

29. The compositions according to claim 27, characterized in that they take a form for topical administration, such as cream, ointment, gels, lotions or patch.

30. The compositions according to claim 27, characterized in that they take a form for parenteral administration, such as a sterile or sterilizable injectable solution.
